(19) United States
(12) Patent Application Publication
(10) Pub. No.: US 2003/0077757 A1
(43) Pub. Date: Apr. 24, 2003

(54) Method of treating aging-related disorders

(76) Inventor: William H. Andrews, Reno, NV (US)

Correspondence Address: BOZICEVIC, FIELD & FRANCIS LLP, 200 MIDDLEFIELD RD, SUITE 200, MENLO PARK, CA, 94025, US

(21) Appl. No.: 10/278,743

(22) Filed: Oct. 21, 2002

Related U.S. Application Data
(63) Continuation-in-part of application No. 09/721,482, filed on Nov. 22, 2000, now abandoned.
(60) Provisional application No. 60/175,575, filed on Jan. 11, 2000.

(51) Int. Cl. C12P 21/08

(52) U.S. Cl. 435/70.21; 435/69.6

(57) ABSTRACT

A method for increasing the proliferative capacity of a cell provides a substance that modulates the activity of a myc-like protein and contacts the cell with the substance. The cell is optionally mammalian. Preferably, the mammalian cell is not a stem cell. In another embodiment, the method utilizes a substance that is a peptide, protein, carbohydrate, small molecule, lipid, or natural extract. Also, the myc-like activity can be modulated by increasing the amount of myc-like protein in the cell. In another embodiment, the amount of myc-like protein is increased by inserting a transgene therefor. Preferably the myc-like protein is L2-myc, N-myc or L-myc. Also disclosed is a method of delaying aging in a cell that includes administering a pharmaceutical composition with L2-myc or a portion thereof which is sufficient to promote telomerase expression; and a pharmaceutically acceptable excipient, whereby the increased telomerase increases the length of telomeres in aging tissues. The method may include increasing telomerase expression in non-expressing tissue. In another embodiment, the L2-myc or a portion thereof is pegylated. Alternatively, the L2-myc or a portion thereof is joined to a molecule to increase its entry into cells. Alternatively, the method provides a compound which interferes with ubiquitination of L2-myc. In a preferred embodiment, L2-myc is provided in a topical dosage form. Also disclosed is a composition for delaying aging in a cell, said composition comprising a small molecule which mimics the activities of L2-myc in promoting expression of telomerase, and a pharmaceutically acceptable excipient.

### CROSS-REFERENCE TO RELATED APPLICATIONS

This application is a continuation-in-part of application Ser. No. 09/721,482 filed on Nov. 22, 2000; which application, pursuant to 35 U.S.C. §119(e), claims priority to the filing date of the U.S. Provisional Patent Application Serial No.: (a) 60/175,575 filed Jan. 11, 2000; the disclosures of which are herein incorporated by reference.

### INTRODUCTION

### Technical Feild

This invention is in the field of pharmaceutical therapy. More specifically, the invention is a method for the prevention and amelioration of age-related disorders.

### Background

Considerable research has been directed to the study of aging in individual cells and in disorders that result from cellular changes associated with aging. Most cells, with the exception of embryonic stem cells, have a limited capacity to divide. As human cells divide, the ends of their chromosomes get shorter. Eventually, a cell whose telomeres have reached some critical length will stop dividing altogether, a state known as senescence (Hayflick and Moorhead, Exp Cell Res 25:585-621, 1961; Goldstein, Science 249:1129-33, 1990).

A recent publication (Bodnar, A. G., M. Ouellette, et al. Science 279(5349): 349-52, 1998.) showed that inserting into normal aging cells the gene for the enzyme telomerase, in a form that expresses telomerase constitutively, caused cells to stop aging. This indicates that the enzyme telomerase is a key to controlling the aging process in cells.

What is needed is an innovative method of treating cells to slow or stop the aging of cells or to revitalize cell systems that have already aged. The present invention addresses this need.

### SUMMARY OF INVENTION

It is an object of this invention to provide a method for modulating the proliferative capacity of a cell. This method includes providing a substance that modulates the activity of a myc-like protein and contacting the cell with the substance. In one embodiment of this method the contacted cell is mammalian, preferably human. In another embodiment of the method, the mammalian cell is not a stem cell. In yet another embodiment of this method, the provided substance is a peptide, protein, carbohydrate, small molecule, lipid, or natural extract. In another embodiment of the method, the myc-like activity is modulated by increasing or decreasing the amount of myc-like protein in the cell. In another embodiment, the amount of myc-like protein is increased by inserting a transgene therefor. Preferably, the transgene encodes L-myc, L2-myc, or N-myc.

Also disclosed is a method of delaying aging in a cell with the step of administering a pharmaceutical composition which comprises L2-myc or a portion thereof which is sufficient to promote telomerase expression and a pharmaceutically acceptable excipient, whereby the increased telomerase increases the length of telomeres in aging tissues.

In another embodiment of the method telomerase expression is increased in non-expressing tissue (i.e., tissue composed of cells that do not normally express telomerase).

In another embodiment of the method, L2-myc or a portion thereof is pegylated. In yet another embodiment of the method, L2-myc or a portion thereof is joined to a molecule to increase its entry into cells. In yet another embodiment of the method, a compound interferes with ubiquitination of L2-myc. Optionally, the method includes a topical dosage form.

Also disclosed is a composition for delaying aging in a cell that includes a small molecule which mimics the activities of L2-myc in promoting expression of telomerase, and a pharmaceutically acceptable excipient.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing the elements that are described in the publications which might be used in connection with the presently described invention.

### DETAILED DESCRIPTION OF INVENTION

The present invention comprises a method for modulating the proliferative capacity of a cell by modulating the activity of certain transcription factors. Transcription factors are a large and growing category of important proteins. These proteins interact with each other and with DNA. The factors either facilitate or inhibit the activity of genes. Transcription factors are recognized as powerful biochemicals having potential to treat a variety of diseases by blocking the deleterious effects of certain disease-causing genes. In humans, transcription is a highly regulated and exquisitely modulated process. A complex apparatus, consisting of as many as 50 unique proteins, enable transcription of the information in the DNA template into RNA. For example, basal transcription requires at least 8 different factors, some of which are composed of several different subunits. There are three classes of transcription factors involved in the regulation of class II genes: basal factors, co-activators and activators-repressors. The promoter is a region on DNA to which RNA polymerase binds and initiates transcription. The promoter facilitates transcription and expression of that gene. The promoter is a region of DNA that typically lies upstream of the transcription initiation site of a gene and controls where and when that gene is expressed. A repressor is the product of a regulatory gene that is a protein that prevents transcription.

The factors responsible for modulating telomerase transcription are not known but have been investigated. Factors among the most studied are members of the myc family. The c-myc protein is a key cell growth regulator and is activated in a large fraction of human malignancies. Besides c-myc, the major members of the families are L-myc and N-myc. The myc proteins contain two regions: an N-terminal transactivation domain and a C-terminal basic helix-loop-helix (bHLH) leucine zipper motif known to mediate dimerization and sequence specific DNA binding. Myc functions as a heterodimer with other bHLH leucine zipper proteins. Max specifically dimerizes with Myc, and Myc-Max heterodimers function as transcriptional activators, binding a hexanucleotide motif called the E-box. Some c-myc is found in a wide variety of developing tissues; whereas, abundant N- and L-myc expression is limited to fewer tissues. (Reviewed by Morgenbesser et al. EMBO J. 14:743-56, 1995). Though the myc family, primarily c-myc, has been studied for nearly two decades, “c-myc remains a fascinating and enigmatic subject [and] its exact function has remained elusive.” (Sakamuro D and Prendergast GC Oncogene 18:2942-54, 1999).

In PCT Publication W099/35243, published Jul. 15, 1999, human mammary epithelial cells, diploid fibroblasts and breast cancer cells were infected with virus supernatants containing mouse c-myc/MarXII-hygro and other plasmids. After mouse c-myc introduction, telomerase activity increased to a level of activity similar to that observed in breast carcinoma cell lines. Specifically, mouse myc increased expression of the catalytic subunit of telomerase (hEST2). This activation of telomerase was sufficient to increase average telomere length and extend lifespans in normal human mammary epithelial cells. It was proposed that mouse c-myc contributes to tumor formation by activating telomerase.

Some have proposed that the telomerase gene be therapeutically controlled with C-myc. C-myc, dimerized with Max, effectively enhanced telomerase expression. However, suitable sites for c-myc to couple to the genome are quite frequent, estimated at a frequency of 1/1,000 bases. Such a protein would not be very specific for telomerase, and indeed c-myc has been shown to affect transcription of many different proteins.

The present invention is based on the discovery that telomerase expression is naturally modulated by a myc-like protein that is not c-myc. L2-myc is not widely expressed in human cells. Testis-specific expression of the human L2-MYC gene was discussed in 1991 by Robertson et al. (Nucleic Acids Res 19(11):3129-37). L2-myc was found to express almost exclusively in human adult normal testes and not in human testis from second-trimester fetuses. This suggests a germ cell rather than somatic cell origin of the transcript and possible developmental regulation of L2-MYC. Germ cells are the only normal human cells that express telomerase. (Robertson et al., ibid.).

L2-myc does not seem to be involved in differentiation or proliferation, even though related c-myc and L-myc have been found in some tumors. For example, in a human small cell lung cancer which over-expresses L-myc, an L-myc antisense DNA inhibited proliferation (Dosaka-Akita et al, Cancer Res 55(7):1559-64, 1995). L-myc also dimerizes with Max to positively regulates transcription of L-myc-specific gene targets and/or modulates the activities of key cellular proteins through interactions mediated by its transcription activation domain (see FIG. 9A of Morgenbesser et al. EMBO J 14(4): 743-56, 1995).

L2-MYC is an intronless gene, which like several other functional intronless genes is expressed in significant amounts only in adult testis. The coding region of L2-MYC is conserved for the leucine zipper and helix-loop-helix motifs which affect transcription regulation by involvement in protein dimerization and sequence-specific DNA binding. The leucine zipper of L2-MYC has only conservative amino acid substitutions from sequences of other myc gene family members and therefore shares the capacity to code for a stable alpha-helical structure with oppositely charged acidic and basic residues which could form ion pairs. Notably, one of five leucine residues in the chain of heptad repeats is substituted by a methionine residue in both L1-MYC and L2-MYC. But methionine is considered the most compatible substitute for leucine in zipper formations on which there should be minimal effect.

The topography of N-myc is strikingly similar to that of c-myc: both genes contain three exons of similar lengths; the coding elements of both genes are located in the second and third exons; and both genes have unusually long 5′ untranslated regions in their mRNAs, with features that raise the possibility that expression of the genes may be subject to similar controls of translation.

Based on the above similarities and differences between L-MYC and other members of the Myc family, it is clear that all are highly conserved and thus capable of forming dimers with MAX. I proposed a method of using a substance which modulates the activity of L-MYC and N-MYC to promote transcription of telomerase, either directly or indirectly. More preferably, L2-MYC is modulated to promote transcription of telomerase.

Direct promotion of telomerase transcription could be performed by the administration of a myc-like protein to promote an anti-aging effect. Administration of L1-myc, L2-myc or N-myc to cells can be performed in a variety of ways detailed below. Indirect promotion of telomerase transcription can be performed by administering a substance which modulates the activity of a myc-like protein in desired cells. Either way, more myc-like protein would be available to combine with MAX to encourage telomerase transcription and promote anti-aging.

Providing a substance that modulates a myc-like protein could transiently induce the expression of the enzyme telomerase and thus have the therapeutic effects of preventing and treating age-related diseases, improving health, and extending lifespan in humans. There is a multitude of aging-related “decline-in-function” diseases that humans can expect to suffer as they grow older. They include osteoporosis, Alzheimer's disease, immune senescence, wrinkled skin, and all diseases brought on by the over-proliferation of cells in the human body. All of these are disease targets for a telomerase-inducing drug. This method of inducing telomerase can also treat premature aging in childhood, known as progeria, as well as the immune senescence outcome of AIDS and Down's syndrome. The method should also improve the human body's ability to protect itself against cancer and heart disease. But, overall, the health and lifespan of humans may be expected to significantly increase by delaying cellular aging.

As used herein, the term “myc-like” refers to members of the myc family but excluding c-myc. Therefore, the use of “myc-like” herein is dissimilar to that of the art in which the term “myc” alone has generally meant c-myc. The term “myc-like” includes but is not limited to L-myc (also known as mycl), a variant L2-myc (or mycl2) and N-myc (or mycn). Preferably the term Myc-like protein comprises L2-myc protein or a portion thereof.

A substance is a chemical that is denoted by its biological activity (in this case, modulating myc-like proteins). The substance may be a large molecule such as a polypeptide, protein, carbohydrate or lipid. The substance also can be a small organic molecule, which is of natural origin or chemically synthesized and generally has a molecular mass less than 1,000. The substance may be highly purified, or it may be a partially purified natural extract that modulates the activity of myc-like proteins. A natural extract as used herein is a substance obtained from a natural source, such as an animal, plant, bacteria, yeast, or a portion thereof, which has not been genetically engineered to produce the substance. Generally, a natural extract is produced by grinding up the base material and separating the substance from the base material with an aqueous or organic solvent. The purity of the substance in the extract can range from 0.1% to 99%, preferably 5-60%, and more preferably at least 20%.

The term “modulates” indicates that there is an increase or decrease in biological activity. Increase in biological activity of a myc-like protein may result from, but is not limited to, the following: activation of the promoter of the gene for a myc-like protein, production of additional RNA therefor, processing of an increased amount of RNA therefor, longer half-life of the mRNA therefor, production of more myc-like protein by the mRNA, longer half-life of the myc-like protein, increased availability of the coactivators of the myc-like protein (e.g., Max), decreased availability of molecules that compete for Max, and decreased competition for the myc binding site.

The term “proliferative capacity” as used herein refers to the ability of eukaryotic cells to continue to divide. Eukaryotic cells have very limited proliferative capacity if their telomeres are short; they can perform only a few divisions; such cells also may be termed senescent. Cells with high proliferative capacity have the ability to divide for many generations and are not senescent.

A substance capable of modulating a myc-like protein also can be used in the culturing of cells for basic research, production of monoclonal antibodies, bone marrow transplants, and skin grafts. Other opportunities include extending lifespan in research animals, household pets, and livestock.

One aspect of the present invention relates to methods and reagents for extending the life span e.g., the number of mitotic divisions of which a cell is capable. Cells may be isolated in culture for part or all of the treatment.

In general, the invention provides a method for increasing the proliferative capacity of metazoan cells, preferably mammalian cells, by contacting the cell with a substance modulating a myc-like protein. Increased activity of a myc-like protein in turn contributes to activation of telomerase activity in the cells. In certain embodiments, the subject method relies on ectopic expression of telomerase. By “ectopic expression” is meant that cells are caused to express, e.g., by expression of a heterologous or endogenous gene or by transcellular uptake of a protein, a higher than normal level of telomerase than the cell normally would, considering its particular phenotype.

Transgenes are “packages” of genetic material (i.e., DNA) that are inserted into the genome of a cell via gene splicing techniques. As appropriate, the transgene may include promoters, leader sequence, termination codon, etc.

In still other embodiments, the subject method can be carried out by contacting the cell with an agent that inhibits degradation of a myc-like protein or its activator; thereby increasing the half-life of the protein. For example, the method can utilize an agent that inhibits ubiquitination, slows destruction of the myc-like protein, and thereby increases the cellular concentration of the myc-like protein. In preferred embodiments, such agents are small, membrane-permeant, organic molecules.

Another method for modulating a myc-like protein is pegylation with polyethylene glycol (PEG) derivatives. Reaction conditions for coupling PEG to a protein vary depending on the protein, the desired degree of PEGylation and the PEG derivative utilized. Factors involved in the choice of PEG derivative include, but are not limited to, desired point of attachment (e.g., lysine vs. cysteine); hydrolytic stability and reactivity of the derivative; stability, toxicity and antigenicity of the linkage; and suitability for analysis. Details on these points are available from the manufacturer of the derivative. The most frequently used derivatives for lysine attachment are the N-hydroxylsuccinimide (NHS) active esters such as PEG succinimidyl succinate (SS-PEG) and succinimidyl propionate (SPA-PEG). Typically, several PEGs can be attached to each protein at pH 8-9.5, room temperature, within 30 minutes, if equal masses of PEG (MW 5,000) and protein are mixed. For some proteins it may be necessary to add a greater mass (e.g., as much as 10 fold) of PEG relative to the protein. Several sulfhydryl-selective PEGs are available, including vinylsulfone, iodoacetamide, maleimide and dithio-orthopyridine. Typical reaction conditions for these derivatives are pH 7-8, slight molar excess of PEG, and 0.5-2 hour reaction at room temperature. For sterically hindered sulfhydryl groups, required reaction times may be significantly longer.

In preferred embodiments, nucleotide sequences can be the substance that modulates a myc-like protein. DNA from some portion of the endogenous gene (exon sequence, intron sequence, promoter sequences, etc.) which directs recombination and heterologous transcriptional regulatory sequence(s) is operably linked to the coding sequence for the genomic gene after recombination of the gene activation construct. The construct may further include a reporter gene to detect the presence of the knockout construct in the cell. Such a gene activation construct is inserted into the cell and integrates with the genomic DNA of the cell in such a position as to provide the heterologous regulatory sequences in operative association with, e.g., the L2-MYC gene. Such insertion occurs by recombination of regions of the activation construct that are homologous to the endogenous L2-MYC gene sequence.

In another embodiment, membrane permeable drugs (e.g., preferably small organic molecules) can be identified which activate the expression of an endogenous myc-like gene. The RNA or genomic sequences of L-myc, L2-myc or N-myc are used to produce reporter constructs in which a reporter gene is operably linked to the transcriptional regulatory sequence of the respective gene. When transfected into cells that possess the appropriate intracellular machinery for activation of the reporter construct through the gene's regulatory sequence, the resulting cells can be used in a cell-based screening approach for identifying such compounds.

When the cells are treated in culture, RNA encoding the myc-like product can be introduced directly into the cell, e.g., from RNA generated by in vitro transcription. In preferred embodiments, the RNA sequence is adapted to that most acceptable to the recipient cell. In addition, the RNA is preferably a modified polynucleotide which is resistant to endogenous nucleases, e.g., exonucleases and endonucleases. Exemplary nucleic acid modifications which can be used to generate such RNA include phosphoramidate, phosphothioate and methylphosphonate analogs of nucleic acids (cf. U.S. Pat. Nos. 5,176,996; 5,264,564; and 5,256,775) or peptide nucleic acids (PNAs).

In another embodiment of the subject method, the myc-like protein can be contacted with a cell under conditions wherein the protein is taken up by the cell, e.g., internalized, without the need for recombinant expression in the cell. For instance, in the application of the subject method to skin, mucosa, and the like, a variety of techniques have been developed for the transcytotic delivery of proteins which are discussed in detail supra.

In one example, the myc-like protein is provided for transmucosal or transdermal delivery. For such administration, penetrants appropriate to the barrier to be permeated are used in the formulation with the polypeptide. Such penetrants are generally known in the art and include, but are not limited to, bile salts and fusidic acid derivatives for transmucosal administration. Detergents may be used to facilitate permeation. Transmucosal administration may be done with nasal sprays or suppositories. For topical administration, the proteins of the invention are formulated into ointments, salves, gels, or creams as generally known in the art. For example, Chien et al. (J Pharm Sci 78:376-83, 1989) describes direct current iontophoretic transdermal delivery of peptide and protein drugs. Srinivasan et al. (J Pharm Sci 78:370-75, 1989) describes transdermal iontophoretic drug delivery. Debs et al. (J Biol Chem 265:10189, 1990) describes the use of cationic lipids for intracellular delivery of biologically active molecules. U.S. Pat. No. 5,190,762 describes methods of administering proteins to living skin cells.

In another embodiment, the myc-like polypeptide is provided as a chimeric polypeptide which includes a heterologous peptide sequence, or internalizing peptide, which drives the translocation of an extracellular form of a therapeutic polypeptide sequence across a cell membrane to facilitate entry of the therapeutic polypeptide. The internalizing peptide, by itself, can cross a cellular membrane by, e.g., transcytosis, at a relatively high rate. This peptide is conjugated or fused to the myc-like protein. The resulting chimeric polypeptide is transported into cells at a higher rate than the myc-like protein alone. The chimeric protein is particularly preferred for topical applications.

Another example of an internalizing peptide is the Drosophila antepennepedia protein or homologs thereof. A 60-amino acid homeodomain of the homeo-protein antepennepedia translocates through biological membranes and facilitates the translocation of polypeptides to which it is coupled (Derossi et al. J Biol Chem 269:10444-10450, 1994; Perez et al. J Cell Sci 102:717-22, 1992). Recently, it has been reported that fragments as small as 16 amino acids of this protein can drive internalization. The present invention contemplates a chimeric protein comprising at least a portion of a myc-like protein and at least a portion of the antepennepedia protein (or homolog thereof) sufficient to increase the transmembrane transport of the chimeric protein, relative to the myc-like protein alone.

Another internalizing peptide is HIV transactivator (TAT) protein which is taken up by cells in tissue culture (Frankel & Pabo, Cell 55:1189-1193, 1989. Moreover, peptide fragments (e.g., residues 37-62) are rapidly taken up by cells (Green and Loewenstein, Cell 55:1179-1188, 1989). A highly basic region mediates internalization and nuclear targeting (Ruben et al. J Virol 63:1-8, 1989) and can be conjugated to the myc-like protein for appropriate targeting. Another transcellular carrier includes a sufficient portion of mastoparat (Higashijima et al, J Biol Chem 265:14176. 1990).

Another method of increasing effective entry of myc-like protein into cells is conjugating the protein to hydrophilic polypeptides, at least a portion of which is capable of crossing the membrane by receptor-mediated transcytosis. Suitable internalizing peptides of this type can be generated from all or a portion of proteins such as a histone, insulin, transferrin, basic albumin, prolactin and insulin-like growth factor I (IGF-I), IGF-II or other growth factors. For instance, an insulin fragment that has affinity for insulin receptors on capillary cells is capable of transmembrane transport. Because this insulin fragment has little effect on blood sugar, it is preferred for use as an internalizing protein over insulin. Other growth factors also have membrane-internalizing portions that can be used in a chimeric protein.

Yet another class of translocating/internalizing peptides binds to membranes in a pH-dependent manner. Some peptides are helical at an acidic pH, because of hydrophobic and hydrophilic interfaces. More specifically, in a pH range of about 5.0 to 5.5, an internalizing peptide forms an alpha-helical, amphiphilic structure that facilitates insertion into the membrane. An alpha-helix-inducing acidic pH may be found, for example, in the low pH within cellular endosomes. Such internalizing peptides can be used to facilitate transport of the myc-like protein to be taken up from endosomal compartments into the cytoplasm.

A preferred pH-dependent membrane-binding internalizing peptide is rich in helix-forming residues such as glutamate, methionine, alanine, and leucine—similar to the myc-like proteins. In addition, a preferred internalizing peptide sequence includes ionizable residues having pKa's in the range of pH 5-7. Thus, there is sufficient uncharged membrane-binding domain in the peptide at pH 5 to allow insertion into the cell membrane.

Yet another preferred pH-dependent membrane-internalizing peptide has been disclosed by Subbarao et al. (Biochemistry 26:2964, 1987). Other preferred internalizing peptides include, but are not limited to, apo-lipoprotein A-1 and B; toxins such as melittin, bombolittin, delta hemolysin and the pardaxins; antibiotics such as alamethicin; hormones such as calcitonin, corticotrophin releasing factor, beta-endorphin, glucagon, parathyroid hormone, pancreatin; and signal sequences of secreted proteins. In addition, internalizing peptides may be modified through attachment of substituents that enhance the alpha-helical character at acidic pH.

Yet another class of internalizing peptides suitable for use with the present invention include hydrophobic domains that are “hidden” at physiological pH, but are exposed in the low pH environment of the target cell endosome. Upon pH-induced unfolding and exposure of the hydrophobic domain, the moiety binds to lipid bilayers and effects translocation of the covalently linked polypeptide into the cell cytoplasm. Such internalizing peptides may be modeled after sequences identified in, for example, Pseudomonas exotoxin A, clathrin or Diphtheria toxin.

Pore-forming proteins or peptides may also serve as internalizing peptides herein, e.g., C9 complement protein, cytolytic T-cell molecules or NK-cell molecules.

The practice of the present invention will employ, unless otherwise indicated, conventional techniques of cell biology, cell culture, molecular biology, transgenic biology, microbiology, recombinant DNA and immunology, which are within the skill of the art. Such techniques are described in the literature. See, for example, MOLECULAR CLONING: A LABORATORY MANUAL, 2d ed. Ed. by Sambrook Fritsch and Maniatis (Cold Spring Harbor Laboratory Press. 1989; DNA CLONING: VOLUMES I AND II. Ed by D. N. Glover, 1985; OLIGONUCLEOTIDE SYNTHESIS. Ed. By M. J. Gait, 1984; Mullis et al., U.S. Pat. No. 4,683,195; NUCLEIC ACID HYBRIDIZATION. Ed by B. D. Hames and S. J. Higgins, 1984; TRANSCRIPTION AND TRANSLATION. Ed. By B. D. Hames and S. J. Higgins, 1984; CULTURE OF ANIMAL CELLS (Ed. By R. I. Freshney, Alan R. Liss, Inc., 1987; IMMOBILIZED CELLS AND ENZYMES, IRL Press, 1986; PRACTICAL GUIDE TO MOLECULAR CLONING, B. Perbal, 1984; GENE TRANSFER VECTORS FOR MAMMALIAN CELLS, Ed by J. H. Miller and M. P. Calos, Cold Spring Harbor Laboratory, 1987; METHODS IN ENZYMOLOGY: VOLS. 154 AND 155, Ed by Wu et al; IMMUNNOCHEMICAL METHODS IN CELL AND MOLECULAR BIOLOGY, Ed by Mayer and Walker, Academic Press, London, 1987; HANDBOOK OF EXPERIMENTAL IMMUNOLOGY: VOLS. I-IV, Ed. By D. M. Weir and C. C. Blackwell, 1986; MANIPULATING THE MOUSE EMBRYO, Cold Spring Harbor Laboratory Press, Cold Spring Harbor, N.Y., 1986.

Reagents for Basic Research

The inventive substances for modulating the activity of a myc-like protein (or myc-modulating substances) are provided as reagents to extend telomere length and replicative capacity in cells cultured for basic research. All somatic cell strains have a limited lifespan (the Hayflick Limit) in culture. The myc-like protein-modulating substances enable growth beyond this natural limit.

Reagents for the Production of Antibodies

Use of a myc-like protein-modulating substance can aid in the process of producing monoclonal antibodies. Monoclonal antibodies are reagents with the ability to bind very specifically to targets known as antigens. This binding specificity makes them potentially powerful therapeutic and diagnostic tools, able to block, trigger, or diagnose a particular biochemical or cytological function.

Ordinarily, production of monoclonal antibodies requires generating the antibody-producing cells and then immortalizing those cells. First, an animal is immunized by exposure to an antigen that elicits antibody formation. Then, antibody-producing B-lymphocytes are isolated from the immunized animal. These B-lymphocytes are next fused with immortalized myeloma cells to produce “hybridomas”, which are immortalized antibody-producing cells. By adding the myc-modulating substance directly to the isolated B-lymphocytes in culture, the additional steps of immortalization with myeloma cells are avoided. Eliminating the immortalization steps should reduce production time and significantly cut production costs. Procedures for long term culturing of primary B-lymphocytes are known in the art (Kumar et al, Immunology Letters 47:193-97, 1995).

In one embodiment of the invention, then, the subject methods are used to immortalize antibody producing cells in culture. Exemplary cells that are modulated according to the present invention include, but are not limited to: antibody producing cells, e.g. B cells and plasma cells which may be isolated and identified for their ability to produce a desired antibody using known technology as, for example, taught in U.S. Pat. No. 5,627,052. These cells may either secrete antibodies (antibody-secreting cells) or maintain antibodies on the surface of the cell without secretion into the cellular environment. Such cells have a limited lifespan in culture, and are usefully immortalized by upregulating expression of telomerase using the methods of the present invention.

Because the above-described methods are methods of increasing expression of TERT and therefore increasing the proliferative capacity and/or delaying the onset of senescence in a cell, they find applications in the production of a range of reagents, typically cellular or animal reagents. For example, the subject methods may be employed to increase proliferation, delay senescence and/or extend the lifetimes of cultured cells. Cultured cell populations having enhanced TERT expression are produced using any of the protocols as described above.

The subject methods find use in the generation of monoclonal antibodies. An antibody-forming cell may be identified among antibody-forming cells obtained from an animal which has either been immunized with a selected substance, or which has developed an immune response to an antigen as a result of disease. Animals may be immunized with a selected antigen using any of the techniques well known in the art suitable for generating an immune response. Antigens may include any substance to which an antibody may be made, including, among others, proteins, carbohydrates, inorganic or organic molecules, and transition state analogs that resemble intermediates in an enzymatic process. Suitable antigens include, among others, biologically active proteins, hormones, cytokines, and their cell surface receptors, bacterial or parasitic cell membrane or purified components thereof, and vital antigens.

As will be appreciated by one of ordinary skill in the art, antigens which are of low immunogenicity may be accompanied with an adjuvant or hapten in order to increase the immune response (for example, complete or incomplete Freund's adjuvant) or with a carrier such as keyhole limpet hemocyanin (KLH).

Procedures for immunizing animals are well known in the art. Briefly, animals are injected with the selected antigen against which it is desired to raise antibodies. The selected antigen may be accompanied by an adjuvant or hapten, as discussed above, in order to further increase the immune response. Usually the substance is injected into the peritoneal cavity, beneath the skin, or into the muscles or bloodstream. The injection is repeated at varying intervals and the immune response is usually monitored by detecting antibodies in the serum using an appropriate assay that detects the properties of the desired antibody. Large numbers of antibody-forming cells can be found in the spleen and lymph node of the immunized animal. Thus, once an immune response has been generated, the animal is sacrificed, the spleen and lymph nodes are removed, and a single cell suspension is prepared using techniques well known in the art.

Antibody-forming cells may also be obtained from a subject which has generated the cells during the course of a selected disease. For instance, antibody-forming cells from a human with a disease of unknown cause, such as rheumatoid arthritis, may be obtained and used in an effort to identify antibodies which have an effect on the disease process or which may lead to identification of an etiological agent or body component that is involved in the cause of the disease. Similarly, antibody-forming cells may be obtained from subjects with disease due to known etiological agents such as malaria or AIDS. These antibody forming cells may be derived from the blood or lymph nodes, as well as from other diseased or normal tissues. Antibody-forming cells may be prepared from blood collected with an anticoagulant such as heparin or EDTA. The antibody-forming cells may be further separated from erythrocytes and polymorphs using standard procedures such as centrifugation with Ficoll-Hypaque (Pharmacia, Uppsula, Sweden). Antibody-forming cells may also be prepared from solid tissues such as lymph nodes or tumors by dissociation with enzymes such as collagenase and trypsin in the presence of EDTA.

Antibody-forming cells may also be obtained by culture techniques such as in vitro immunization. Briefly, a source of antibody-forming cells, such as a suspension of spleen or lymph node cells, or peripheral blood mononuclear cells are cultured in medium such as RPMI 1640 with 10% fetal bovine serum and a source of the substance against which it is desired to develop antibodies. This medium may be additionally supplemented with amounts of substances known to enhance antibody-forming cell activation and proliferation such as lipopolysaccharide or its derivatives or other bacterial adjuvants or cytokines such as IL-1, IL-2, IL-4, IL-5, IL-6, GM-CSF, and IFN-γ. To enhance immunogenicity, the selected antigen may be coupled to the surface of cells, for example, spleen cells, by conventional techniques such as the use of biotin/avidin as described below.

Antibody-forming cells may also be obtained from very early monoclonal or oligoclonal fusion cultures produced by conventional hybridoma technology. The present invention is advantageous in that it allows rapid selection of antibody-forming cells from unstable, interspecies hybridomas, e.g., formed by fusing antibody-forming cells from animals such as rabbits, humans, cows, pigs, cats, and dogs with a murine myeloma such NS-1.

Antibody-forming cells may be enriched by methods based upon the size or density of the antibody-forming cells relative to other cells. Gradients of varying density of solutions of bovine serum albumin can also be used to separate cells according to density. The fraction that is most enriched for desired antibody-forming cells can be determined in a preliminary procedure using the appropriate indicator system in order to establish the antibody-forming cells.

The identification and culture of antibody producing cells of interest is followed by enhancement of TERT expression is these cells by the subject methods, thereby avoiding the need for the immortalization/fusing step employed in traditional hybridoma manufacture protocols. In such methods, the first step is immunization of the host animal with an immunogen, typically a polypeptide, where the polypeptide will preferably be in substantially pure form, comprising less than about 1% contaminant. The immunogen may comprise the complete protein, fragments or derivatives thereof. To increase the immune response of the host animal, the protein may be combined with an adjuvant, where suitable adjuvants include alum, dextran, sulfate, large polymeric anions, oil & water emulsions, e.g. Freund's adjuvant, Freund's complete adjuvant, and the like. The protein may also be conjugated to synthetic carrier proteins or synthetic antigens. A variety of hosts may be immunized to produce the subject antibodies. Such hosts include rabbits, guinea pigs, rodents (e.g. mice, rats), sheep, goats, and the like. The protein is administered to the host, usually intradermally, with an initial dosage followed by one or more, usually at least two, additional booster dosages. Following immunization, generally, the spleen and/or lymph nodes of an immunized host animal provide a source of plasma cells. The plasma cells are treated according to the subject invention to enhance TERT expression and thereby, increase the proliferative capacity and/or delay senescence to produce “pseudo” immortalized cells. Culture supernatant from individual cells is then screened using standard techniques to identify those producing antibodies with the desired specificity. Suitable animals for production of monoclonal antibodies to a human protein include mouse, rat, hamster, etc. To raise antibodies against the mouse protein, the animal will generally be a hamster, guinea pig, rabbit, etc. The antibody may be purified from the cell supernatants or ascites fluid by conventional techniques, e.g. affinity chromatography using RFLAT-1 protein bound to an insoluble support, protein A sepharose, etc.

In an analogous fashion, the subject methods are employed to enhance TERT expression in non-human animals, e.g., non-human animals employed in laboratory research. Using the subject methods with such animals can provide a number of advantages, including extending the lifetime of difficult and/or expensive to produce transgenic animals. As with the above described cells and cultures thereof, the expression of TERT in the target animals may be enhanced using a number of different protocols, including the administration of an agent that inhibits SC repression and/or targeted disruption of the repressor binding site. The subject methods may be used with a number of different types of animals, where animals of particular interest include mammals, e.g., rodents such as mice and rats, cats, dogs, sheep, rabbits, pigs, cows, horses, and non-human primates, e.g. monkeys, baboons, etc.

Transplantation and Gene Therapy

The inventive myc-like protein-modulating substances are useful for bone marrow transplants for the treatment of cancer and skin grafts for burn victims. In these cases, cells are isolated from a human donor and then cultured for transplantation back into human recipients. During the cell culturing, the cells normally age and senesce, decreasing their useful lifespans. Bone marrow cells, for instance, lose approximately 40% of their replicative capacity during culturing. This problem is aggravated when the cells are first genetically engineered (Decary, Mouly et al. Hum Gene Ther 7(11):1347-50, 1996). In such cases, the therapeutic cells must be expanded from a single engineered cell. By the time there are sufficient cells for transplantation, the cells have undergone the equivalent of 50 years of aging (Decary, Mouly et al. Hum Gene Ther 8(12):1429-38, 1997). The use of a substance to modulate a myc-like protein spares the replicative capacity of bone marrow cells and skin cells during culturing and expansion and thus should significantly improve the survival and effectiveness of bone marrow and skin cell transplants. Any transplantation technology requiring cell culturing should benefit from such a product.

Progeria

Progeria or Hutchinson-Gilford syndrome (Goldstein, N Engl J Med 285(20):1120-9, 1971) is a disease of shortened telomeres for which no known cure exists. It afflicts children, who seldom live past their early twenties. In many ways progeria parallels aging itself. However, these children are born with short telomeres. Their telomeres don't shorten at a faster rate; they are just short to begin with (Allsopp, Vizier et al. Proc Natl Acad Sci USA 89(21):10114-8, 1992). A substance to modulate a myc-like protein may benefit children with this condition.

Immune Senescence

The effectiveness of the immune system decreases with age (Effros and Pawaelec, Immunol Today 18(9):450-4, 1997). Part of this decline is due to fewer T-lymphocytes in the system, a result of lost replicative capacity. Many of the remaining T-lymphocytes experience loss of function as their telomeres shorten and they approach senescence. The inventive substance that modulates a myc-like protein should inhibit immune senescence due to telomere loss. Because aging immune systems are at greater risk of developing pneumonia, cellulitis, influenza, and many other infections, such a substance should reduce morbidity and mortality due to infections.

A special case of immune dysfunction occurs in AIDS (O'Brien, Hosp Pract (Off Ed) 33(7):53-6, 1998). HIV, the virus that causes AIDS, invades white blood cells, particularly CD4 lymphocyte cells, and causes them to reproduce high numbers of the HIV virus, ultimately killing cells. In response to the loss of immune cells (typically about a billion per day), the body produces more CD8 cells to be able to suppress infection. This rapid cell division accelerates telomere shortening, ultimately hastening immune senescence of the CD8 cells. Anti-retroviral therapies have successfully restored the immune systems of AIDS patients, but survival depends upon the remaining fraction of the patient's aged T-cells. Once shortened, telomere length has not been naturally restored within cells. A substance capable of modulating a myc-like protein could restore this length and/or prevent further shortening. Such a substance can spare telomeres and is useful to coadminister with the anti-retroviral treatments currently available for HIV. The substance that modulates a myc-like protein is not expected to destroy the HIV virus; but it may prevent senescence of the CD8 cells, prevent infections, and decrease morbidity and mortality.

Cardiovascular Disease

The inventive substance which modulates a myc-like protein is useful as a cardiovascular drug, because it can extend telomere length and replicative capacity of endothelial cells lining of blood vessel walls (DeBono, Heart 80:110-1, 1998). Endothelial cells form the inner lining of blood vessels and divide and replace themselves in response to stress. Stresses include high blood pressure, excess cholesterol, inflammation, and flow stresses at forks in vessels. As endothelial cells age and can no longer divide sufficiently to replace lost cells, areas under the endothelial layer become exposed. Exposure of the underlying vessel wall increases inflammation, the growth of smooth muscle cells, and the deposition of cholesterol. As a result, the vessel narrows and becomes scarred and irregular, which contributes to even more stress on the vessel (Cooper, Cooke and Dzau, J Gerontol Biol Sci 49:191-6, 1994).

Aging endothelial cells also produce altered amounts of trophic factors (hormones that affect the activity of neighboring cells). These too contribute to increased clotting, proliferation of smooth muscle cells, invasion by white blood cells, accumulation of cholesterol, and other changes, many of which lead to plaque formation and clinical cardiovascular disease (Ibid.).

The inventive substance that modulates a myc-like protein can extend endothelial cell telomeres and help combat the stresses contributing to vessel disease. Many heart attacks may be prevented if endothelial cells were enabled to continue to divide normally and better maintain cardiac vessels. The occurrence of strokes caused by the aging of brain blood vessels may also be significantly reduced by a myc-like protein-modulating substance that helps endothelial cells in the brain blood vessels to continue to divide and perform their intended function.

Skin Rejuvenation

The skin is the first line of defense of the immune system and shows the most visible signs of aging (West, Arch Dermatol 130(1):87-95, 1994). As skin ages, it thins, develops wrinkles, discolors, and heals poorly. Skin cells divide quickly in response to stress and trauma; but, over time, there are fewer and fewer actively dividing skin cells. Compounding the loss of replicative capacity in aging skin is a corresponding loss of support tissues. The number of blood vessels in the skin decreases with age, reducing the nutrients that reach the skin. Also, aged immune cells less effectively fight infection. Nerve cells have fewer branches, slowing the response to pain and increasing the chance of trauma. In aged skin, there are also fewer fat cells, increasing susceptibility to cold and temperature changes. Old skin cells respond more slowly and less accurately to external signals. They produce less vitamin D, collagen, and elastin, allowing the extracellular matrix to deteriorate. As skin thins and loses pigment with age, more ultraviolet light penetrates and damages skin. To repair the increasing ultraviolet damage, skin cells need to divide to replace damaged cells, but aged skin cells have shorter telomeres and are less capable of dividing (Fossel, REVERSING HUMAN AGING. William Morrow & Company, New York City, 1996).

The inventive substance that modulates a myc-like protein is provided in a skin product, preferably applied topically, extends telomere length, and can slow the downward spiral that skin experiences with age. Such a product not only helps protect a person against the impairments of aging skin; it also may permit rejuvenated skin cells to restore youthful immune resistance and appearance. The substance that modulates a myc-like protein can be used for both medical and cosmetic applications. It is important to note that although there are other available treatments for skin that address the loss of particular nutrients or proteins (such as moisturizers and products like Retin-A), the inventive substance that modulates a myc-like protein is needed to address the underlying cause of skin aging—telomere loss.

Historically, topical preparations have been complex mixtures. The compositions of this invention may contain other ingredients conventionally used in the art of skin care compositions, including but not limited to preservatives, preservative enhancers, and active ingredients in addition to the primary active substance. Any optional ingredients must be compatible with the substance that modulates a myc-like protein, such that the activity of the substance does not decrease unacceptably, preferably not to any significant extent, over a useful period (preferably at least about two years under normal storage conditions). For example, if strong oxidizing agents are incompatible with the inventive substance, such agents are avoided.

The compositions of the subject invention may optionally comprise other active ingredients capable of functioning in different ways to enhance the benefits of the primary active myc-modulating substance and/or to provide other benefits. Examples of such substances include, but are not limited to, anti-inflammatory agents, antimicrobial agents, anti-androgens, sunscreens, sunblocks, anti-oxidants/radical scavengers, chelators, depilation agents, desquamation agents, organic hydroxy acids, and natural extracts.

The compositions of the present invention may also include a natural extract of yeast, rice bran or the like such as are known in the art. Such extracts may enhance the skin appearance benefits of the present invention, and are preferably used in an amount of from 0.1% to about 20%, more preferably 0.5% to about 10%, also from 1% to about 5%. A natural extract of yeast is preferred.

Osteoporosis

Two types of cells interplay in osteoporosis: osteoblasts make bone and osteoclasts destroy it. Normally, the two are in balance and maintain a constant turnover of highly structured bone. In youth, bones are resilient, harder to break, and heal quickly. In old age, bones are brittle, break easily, and heal slowly and often improperly. Bone loss has been postulated to occur because aged osteoblasts, having lost much of their replicative capacity, cannot continue to divide at the rate necessary to maintain balance (Hazzard et al. PRINCIPLES OF GERIATRIC MEDICINE AND GERONTOLOGY, 2d ed. McGraw-Hill, New York City, 1994).

Providing the inventive substance that modulates a myc-like protein can lengthen telomeres of osteoblast and osteoclast stem cells, thereby encouraging bone replacement and proper remodeling and reinforcement. Stronger bone would improve the quality of life for the many sufferers of osteoporosis and provide savings from fewer fracture treatments. The inventive substance that modulates a myc-like protein needs to be part of a coordinated treatment effort, as other factors exacerbate osteoporosis. These factors include inadequate calcium uptake, lack of exercise, and reduced estrogen. Thus, a myc-modulating substance needs to be part of a comprehensive treatment regime which also includes calcium, estrogen and exercise.

L2-myc Binds the Telomerase Promoter

Binding of L2-myc to the telomerase promoter is demonstrated by mobility shift DNA-binding assays according to the technique described in Revizin (Biotechniques 7(4):246-55, 1989). Nuclear extracts are prepared from embryonic stem cells. The presence of L2-myc is confirmed by means of L2-myc-specific antibodies, prepared according to techniques well known in the art. See for example, Ausubel et al. SHORT PROTOCOLS IN MOLECULAR BIOLOGY, 3d ed., John Wiley & Sons, 1995.

L2-myc Expression Induces Telomerase Expression

Retroviral vectors containing the L2-myc coding sequence (Morton et al. Genomics 4:367-75, 1989) are used to transfect normal human telomerase-negative cells, such as IMR90 (available from ATCC), using standard techniques (see, for example, Ausubel, supra). Telomerase expression is then detected by the telomere repeat amplification protocol (TRAP) (Oncor, Gaithersburg, Md.).

The myc-like protein binding sites are reduced to their smallest functional forms by standard recombinant DNA techniques. Once the smallest functioning unit of DNA is identified, this DNA is used in protein/DNA binding studies. This can be done by first preparing nuclear extracts of normal and immortal cells. These extracts can be further purified by various methods and fractions can be assayed by DNA retardation gels. Regions of the promoter that proteins bind to are then identified using these extracts in DNA footprinting experiments.

Compounds to mimic myc-like interaction with Max to affect telomerase are identified by high throughput screening methods.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method of producing an antibody, comprising the steps of:
isolating a cell that produces an antibody of interest;
immortalizing said cell by contacting said cell with a substance that modulates the activity of a myc-like protein; and
growing said immortalized cell under conditions which allow the cell to produce the antibody of interest.

2. The method of claim 1, further comprising:
separating away the antibody of interest from said antibody producing cell.

3. The method according to claim 1, wherein said cell is a mammalian cell.

4. The method according to claim 3, wherein said mammalian cell is a B cell or its progeny.

5. The method according to claim 4, wherein said method further comprises contacting a host mammal with an immunogen and then isolating said B cell or its progeny from said contacted host mammal.

6. The method according to claim 5, wherein said host mammal is a mouse.

7. The method according to claim 5, wherein said host mammal is a human.

8. The method according to claim 1, wherein said antibody is a monoclonal antibody.

9. The method according to claim 1, wherein the myc-like protein is L2-myc, N-myc or L-myc.

10. The method according to claim 9, wherein the myc-like protein is L2-myc.

11. A method of producing an antibody, comprising the steps of:
isolating a cell that produces an antibody of interest;
immortalizing said cell by contacting said cell with a substance that modulates the activity of L2-myc; and
growing said immortalized cell under conditions which allow the cell to produce the antibody of interest.

12. The method of claim 11, further comprising:
separating away the antibody of interest from said antibody producing cell.

13. The method according to claim 11, wherein said cell is a mammalian cell.

14. The method according to claim 13, wherein said mammalian cell is a B cell or its progeny.

15. The method according to claim 14, wherein said method further comprises contacting a host mammal with an immunogen and then isolating said B cell or its progeny from said contacted host mammal.

16. The method according to claim 15, wherein said host mammal is a mouse.

17. The method according to claim 15, wherein said host mammal is a human.

18. The method according to claim 11, wherein said antibody is a monoclonal antibody.

19. The method according to claim 1, wherein said agent increases the activity of L2-myc.

20. The method according to claim 19, wherein said agent L2-myc or a nucleic acid encoding the same.
